(19) United States
(12) Patent Application Publication
(10) Pub. No.: US 2022/0340856 A1
(43) Pub. Date: Oct. 27, 2022

(54) Container w/ Heat Plate

(71) Applicant: BIOFRIDGE INC., Escondido, CA (US)

(72) Inventor: Jay Black, Escondido, CA (US)

(73) Assignee: BIOFRIDGE INC., Escondido, CA (US)

(21) Appl. No.: 17/503,591

(22) Filed: Oct. 18, 2021

Related U.S. Application Data
(60) Provisional application No. 63/179,086, filed on Apr. 23, 2021.

(51) Int. Cl. C12M 1/00; A01N 1/02

(52) U.S. Cl. CPC C12M 41/14; C12M 23/22; C12M 23/38; C12M 23/52; A01N 1/0252

(57) ABSTRACT

A platelet incubator usable as a lid for a refrigerated container includes a heating plate and a transparent cover. The heating plate has an aluminum top plate, an aluminum middle plate with an opening into which a silicon mat heater is placed, and a 5052 aluminum alloy bottom plate, with an aperture through which the wires providing electrical energy to the silicon mat heater are placed. The temperature is continuously monitored, and a log is kept for printout, allowing the platelet incubator to meet relevant compliance requirements. An alarm is activated if the temperature of the platelets exceeds twenty-three degrees Celsius, or falls under twenty-one degrees Celsius.

### RELATED APPLICATION

This application claims priority to U.S. Provisional Patent Application Ser. No. 63/179,086 for a “Platelet Incubating Lid,” filed Apr. 23, 2021, and currently co-pending, the entirety of which is incorporated herein by reference.

### FIELD OF THE INVENTION

The present invention pertains generally to a container with a heating mat for use in maintaining items at a desired temperature. More particularly, the present invention pertains to an incubator with a transparent cover. The Present invention is particularly, but not exclusively, useful as a platelet incubator that functions as a lid for another container.

### BACKGROUND OF THE INVENTION

Platelets are small, plate-shaped blood cell fragments that form clots to plug bleeding vessels. Platelets are produced by megakaryocytes in bone marrow and have a life span of several days. A healthy person generally has between 150,000 and 450,000 platelets per microliter of blood.

An abnormally high concentration of platelets, referred to as thrombocytosis, is usually not serious, but severe cases (thrombocythemia) may result in spurious clotting, increasing the risk of thrombosis, embolisms, heart attack, and stroke. Thrombocytosis may be treated with aspirin, or, in extreme cases by platelet pheresis.

Thrombocytopenia, an abnormally low platelet count, is often treated indirectly by focusing on the underlying cause. However, it is common to attempt to mitigate the effects of thrombocytopenia, when a patient has a low platelet count, with a platelet transfusion prior to insertion of a central venous catheter (CVC) in connection with a procedure such as chemotherapy. Platelet transfusion is also practiced in some other cases, such as bone marrow failure.

Platelets collected for transfusion need to be kept warm—between twenty (20) and twenty-four (24) degrees Celsius—and should be subject to gentle agitation in order to avoid damaging settled platelets through hypoxia.

Fresh frozen plasma is stored at negative eighteen (−18) degrees Celsius, and thawed at between one (1) and six (6) degrees Celsius. Cryoprecipitate, which can be obtained from thawed fresh frozen plasma, can be frozen, but after thawing is kept at the same temperature as platelets (20-24 degrees Celsius) until used in a transfusion. Red blood cells are stored at between one (1) and six (6) degrees Celsius.

With any type of transfusion, detailed records must be kept. Identification of the patient to ensure that the correct blood product is administered to the correct patient should be performed not only when the blood product is requested, but again immediately before the transfusion.

### SUMMARY OF THE INVENTION

Disclosed is a platelet incubator designed for use as a lid for a refrigerated container. The platelet incubator includes a heating plate and a transparent cover.

A preferred embodiment of the heating plate has an aluminum top plate, an aluminum middle plate with an opening into which a silicon mat heater is placed, and a 5052 aluminum alloy bottom plate, with an aperture through which the wires providing electrical energy to the silicon mat heater are placed.

The temperature is continuously monitored, and a log is kept for printout, allowing the platelet incubator to meet relevant compliance requirements. An alarm is activated if the temperature of the platelets exceeds twenty-three (23) degrees Celsius, or falls under twenty-one (21) degrees Celsius.

When used as the lid for a refrigerated container, the platelet incubator allows blood products that need to be kept warm, such as platelets and cryoprecipitate, to be stored next to other blood products.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a top perspective view of a preferred embodiment of a platelet incubating lid;

FIG. 2 is front view of the platelet incubating lid;

FIG. 3 is a left side view of the platelet incubating lid;

FIG. 4 is a right side view of the platelet incubating lid;

FIG. 5 is a rear view of the platelet incubating lid;

FIG. 6 is a bottom view of the platelet incubating lid;

FIG. 7 is a top perspective view of the platelet incubating lid showing the transparent cover open;

FIG. 8 is a top perspective view of the platelet incubating lid showing the lid in an open configuration;

FIG. 9 is a top perspective view of the heating plate of the platelet incubating lid;

FIG. 10 is an exploded view of the heating plate of the platelet incubating lid showing its components;

FIG. 11 is an illustration of a printout of a temperature log of the platelet incubator of the platelet incubating lid; and

FIG. 12 is a top perspective view of a preferred embodiment of a platelet incubating lid mounted on top of a portable refrigeration unit.

### DETAILED DESCRIPTION

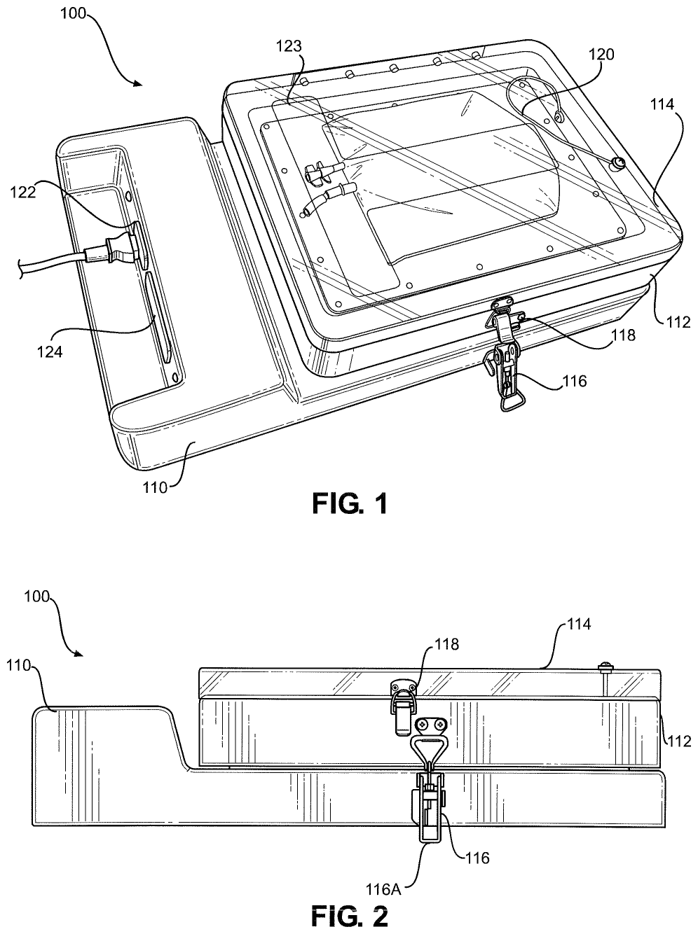

Referring initially to FIG. 1, a top perspective view of a preferred embodiment of a platelet incubating lid 100 is shown. Incubating lid 100 has a base 110 for securing incubating lid 100 to a container, and a heated lid portion 112 with a transparent cover 114. Lid portion 112 is secured to the base 110 in a closed configuration with latch 116, and the transparent cover 114 is secured in a closed configuration to the lid portion 112 with latch 118. Overextension of transparent cover 114 in its open configuration is prevented by a cord 120. A connector 122 allows the incubating lid 100 to be connected to a power source. In a preferred embodiment, connector 122 receives a power cable that connects to a standard one-hundred-twenty (120) volt AC power outlet.

In a preferred embodiment, a pocket 123 located on the upper surface of transparent cover 114 allows for the insertion of identifying documents for the patient for which blood products in the incubating lid 100 and any container to which it is attached are intended.

Referring now to FIG. 2, a front view of platelet incubating lid 100 is shown, illustrating latch 116 and latch 118 secured to hold lid portion 112 and transparent cover 114 in their closed configurations. Latch 116 terminates in a crown 116A that extends below the base 110.

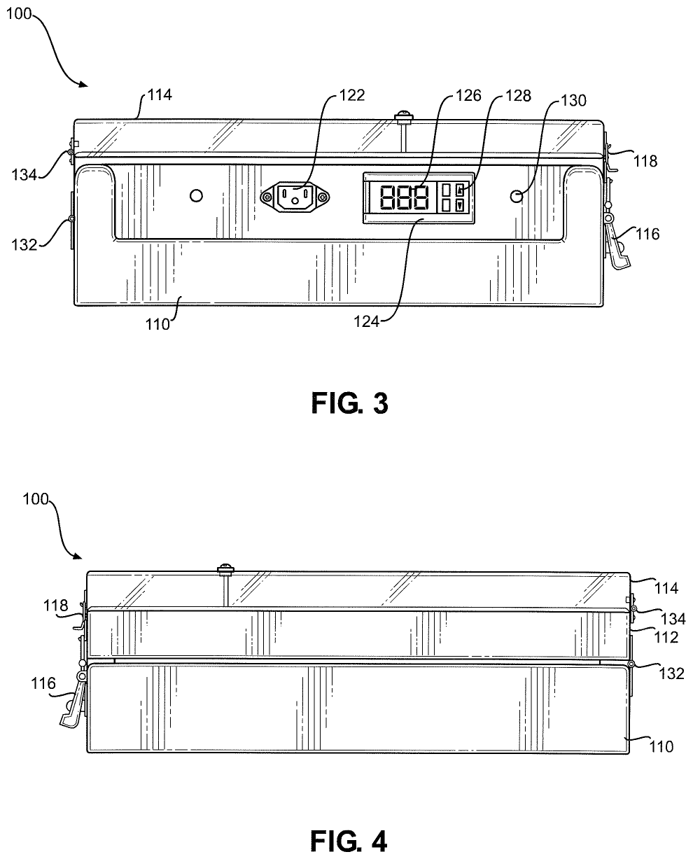

Referring now to FIG. 3, a left side view of platelet incubating lid 100 is shown. In addition to connector 122, incubating lid 100 has a panel 124 with a display 126 to show the incubator temperature and buttons 128 to select the desired incubator temperature. An indicator 130, in the form of a light-emitting diode (LED) in a preferred embodiment, indicates when the incubating lid 100 is in operation. An audible alarm sounds if the incubator temperature exceeds a predetermined upper limit or falls below a predetermined lower limit; the default upper limit is twenty-three (23) degrees Celsius, and the default lower limit is twenty-one (21) degrees Celsius, but these limits can be user adjusted.

Referring now to FIG. 4, a right side view of platelet incubating lid 100 is shown. Hinges 132 connect lid portion 112 to base 110, allowing lid portion 112 to be opened and closed between a closed configuration and an open configuration with respect to base 110. Likewise, hinge 134 connects transparent cover 114 to lid portion 112 allowing transparent cover 114 to be opened and closed between a closed configuration and an open configuration with respect to lid portion 112.

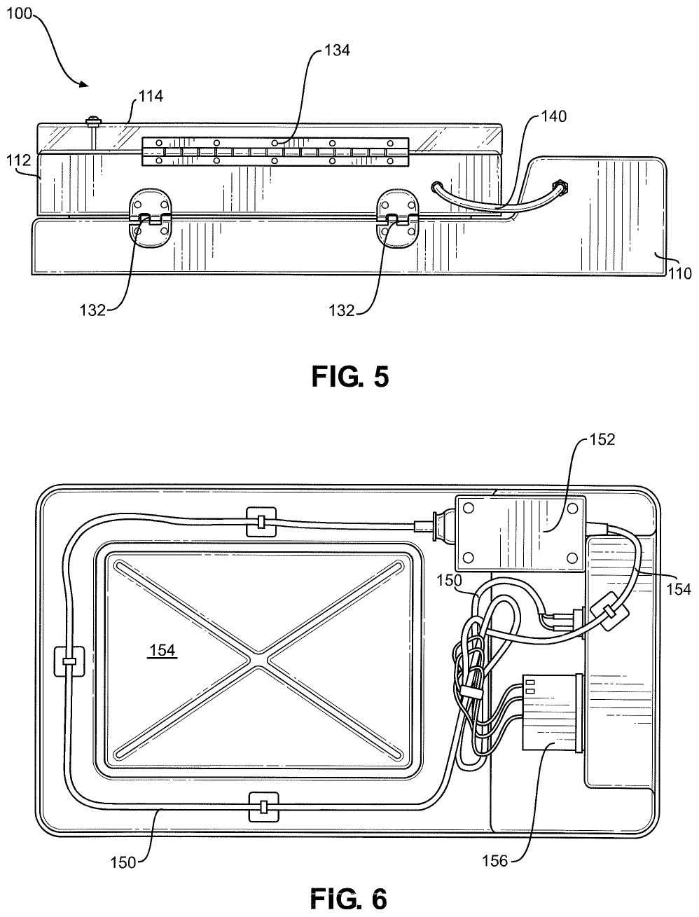

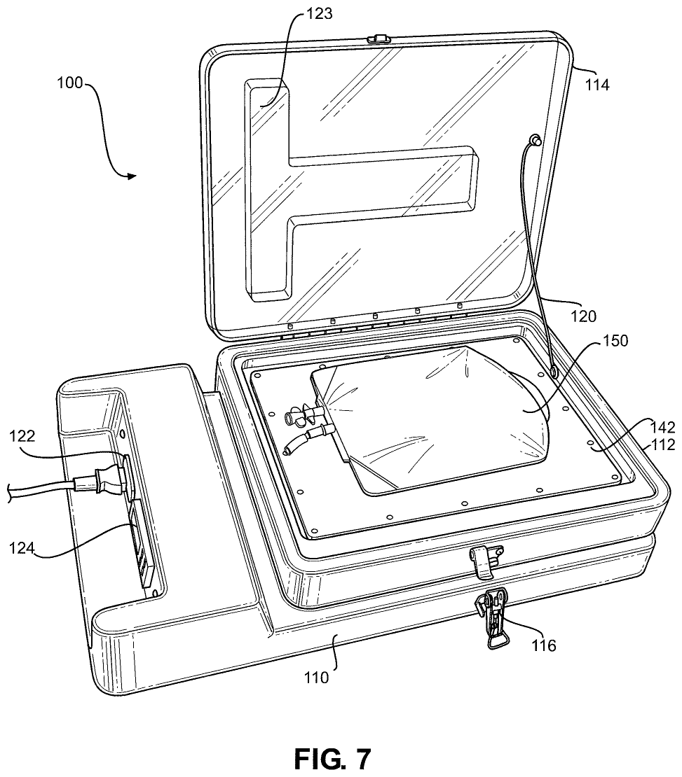

Referring now to FIG. 5, a rear view of platelet incubating lid 100 is shown. Cable 140 extending between base 110 and lid portion 112 includes wires putting panel 124 into electrical communication with heating plate 142 (shown in FIG. 7) in order to provide power to heating plate 142 and obtain and log incubator temperature measurements.

Referring now to FIG. 6, a bottom view of platelet incubating lid 100 is shown. Cable 150 provides power from connector 122 to power supply 152, which includes a transformer to provide the appropriate voltage and current through cable 154 to controller 156. Panel 124 forms the primary user interface mechanism for controller 156, which provides power and temperature control to heating plate 142 (shown in FIG. 7). In a preferred embodiment, controller 156 includes an integrated temperature logger that keeps a record of the incubator temperature. In an alternative embodiment, a separate controller 156 and temperature logger are used.

Referring now to FIG. 7, a perspective view of platelet incubating lid 100 is shown, illustrating the transparent cover 114 in an open configuration and a platelet bag 150 placed on heating plate 142. Lid portion 112 is depicted still in a closed configuration.

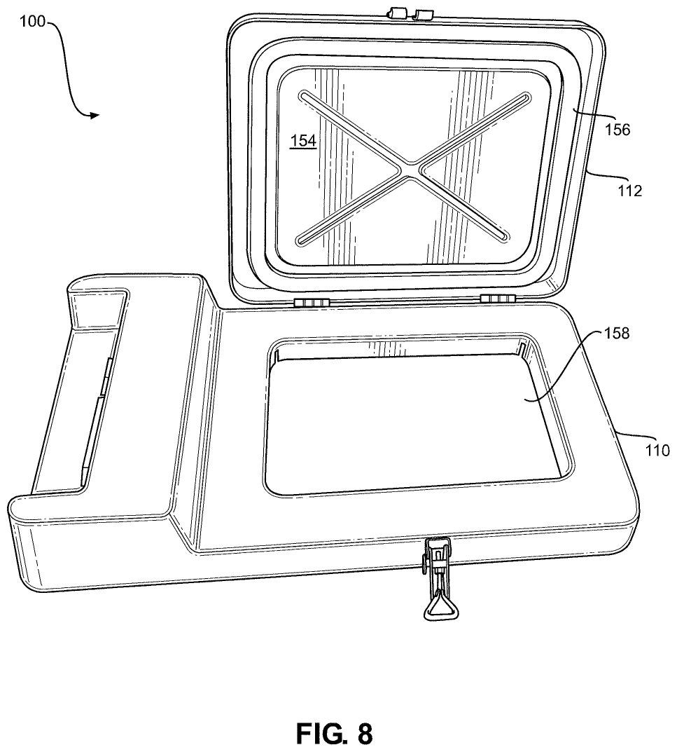

Referring now to FIG. 8, a perspective view of platelet incubating lid 100 is shown, illustrating lid portion 112 in an open configuration. Lid portion 112 has a bottom surface 154 with an insulating gasket 156 around its perimeter, preventing direct contact between base 110 and bottom surface 154 when lid portion 112 is in its closed configuration. Base 110 includes an aperture 158 below the lid portion, allowing access to the contents of a container under the platelet incubating lid 100.

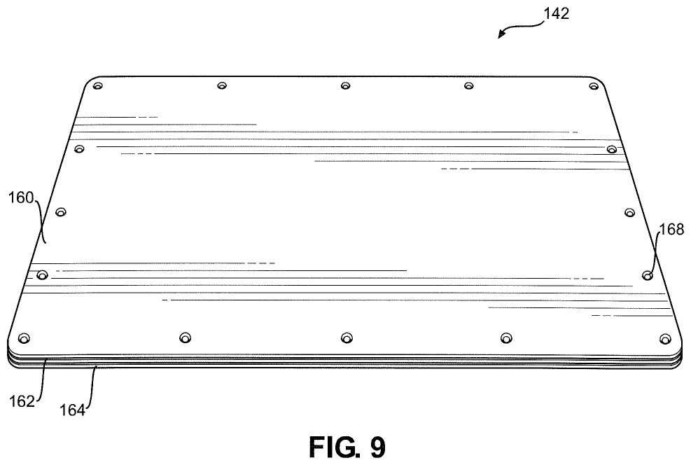

Referring now to FIG. 9, a perspective view of heating plate 142 is shown. In a preferred embodiment, heating plate 142 has three separate aluminum plates, including a top plate 160, a middle plate 162, and a bottom plate 164 joined together and to the lid portion 112 with screws through openings 168.

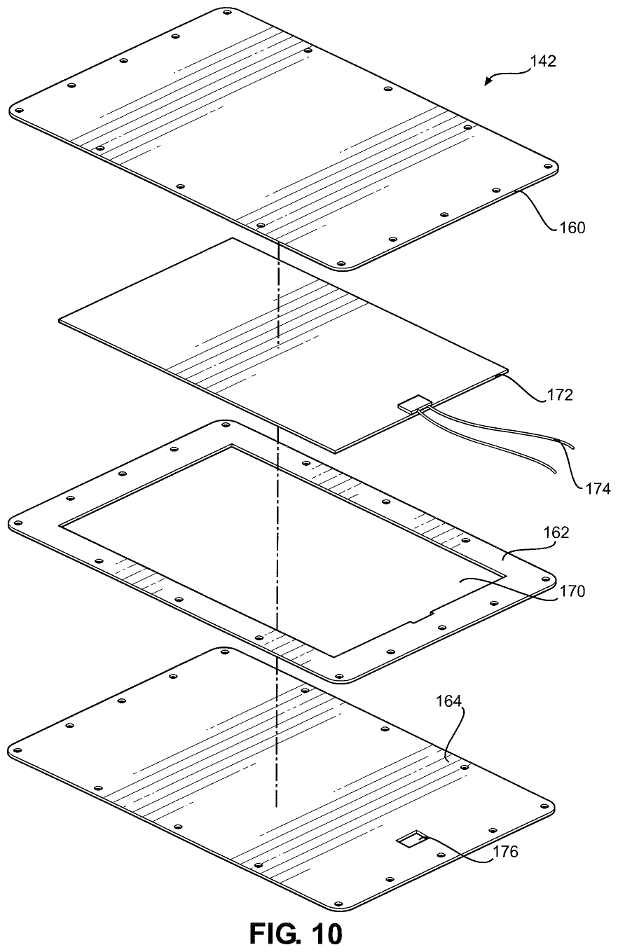

Referring now to FIG. 10, an exploded view of heating plate 142 is shown, further illustrating its structure. Middle plate 162 has an opening 170 into which is seated a silicon mat heater 172. Wires 174 provide power and temperature control and monitoring to mat heater 172, and fit through the smaller aperture 176 in bottom plate 164 when heating plate 142 is fully assembled.

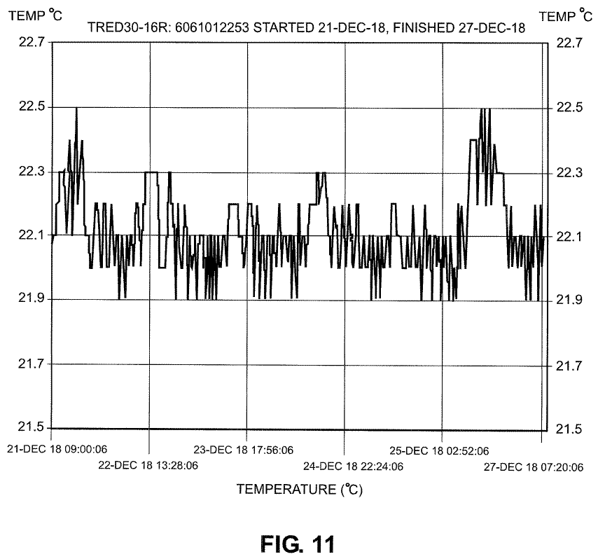

Referring now to FIG. 11, an exemplary graph of a temperature log of a platelet incubating lid 100 is illustrated. The graph displays a continuous log of the incubator temperature during use maintaining the temperature of platelets, cryoprecipitate, or other blood products. Data from the temperature log can be retrieved through the use of a Universal Serial Bus (USB) solid state drive, USB hard disk, solid state memory card, other portable digital storage device, wireless network connection, Bluetooth, or other method known in the art.

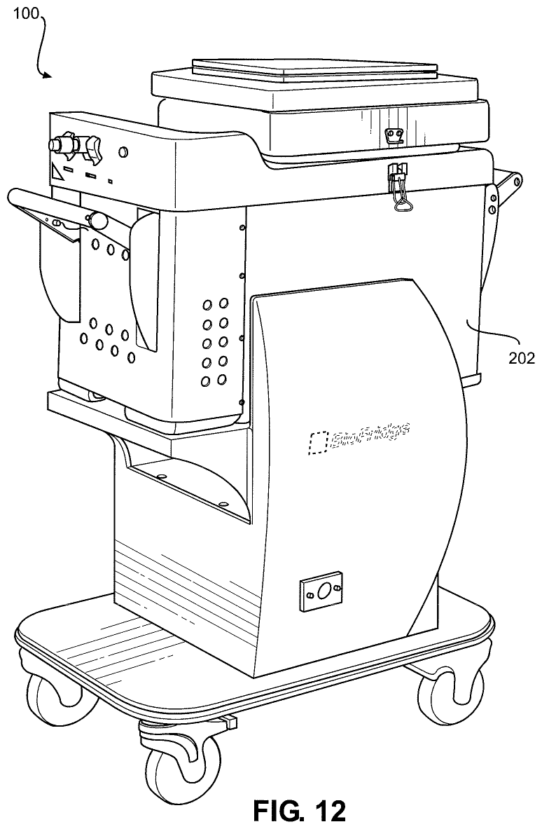

Referring now to FIG. 12, a platelet incubating lid 100 is shown mounted onto a container, and more particularly, on a portable refrigeration unit 202. By mounting platelet incubating lid 100 onto portable refrigeration unit 202, platelets and cryoprecipitate can be stored alongside other blood products, such as red blood cells and plasma, which are kept at lower temperatures.

While there have been shown what are presently considered to be preferred embodiments of the present invention, it will be apparent to those skilled in the art that various changes and modifications can be made herein without departing from the scope and spirit of the invention.

What is claimed is:

1. A platelet incubating lid, comprising:
a platelet incubator having a heating plate and a transparent cover,
wherein the platelet incubator is configured to operate as a lid for a refrigerated container.

2. The platelet incubating lid of claim 1, wherein the heating plate comprises an aluminum top plate, an aluminum middle plate having an opening, a silicon mat heater in the opening of the aluminum middle plate, and an aluminum alloy bottom plate.

3. The platelet incubating lid of claim 1, further comprising a document pocket on an upper surface of the transparent cover.

4. The platelet incubating lid of claim 1, further comprising a panel having a display configured to show a platelet incubator temperature and buttons configured to allow an operator to select a desired platelet incubator temperature.

5. The platelet incubating lid of claim 4, further comprising a light-emitting diode that indicates when the platelet incubator is in operation.

6. The platelet incubating lid of claim 4, wherein the platelet incubator emits an audible alarm if the platelet incubator temperature exceeds a predetermined upper limit or falls below a predetermined lower limit.

7. The platelet incubating lid of claim 1, further comprising a temperature logger configured to keep a record of temperature of the platelet incubator.

8. A platelet incubating lid, comprising:
a base configured for attachment to a container;
a heated lid portion secured to the base; and
a transparent cover secured to the heated lid portion,
wherein the base comprises an aperture that allows access to the contents of the container when the heated lid portion is in an open configuration, and
wherein the heated lid portion covers the aperture of the base while in a closed configuration.

9. The platelet incubating lid of claim 8, wherein the heated lid portion comprises bottom surface having a perimeter and an insulating gasket around the perimeter.

10. The platelet incubating lid of claim 8, wherein the heated lid portion comprises a heating plate.

11. The platelet incubating lid of claim 10, wherein the heating plate comprises an aluminum top plate, an aluminum middle plate having an opening, a silicon mat heater in the opening of the aluminum middle plate, and an aluminum alloy bottom plate.

12. The platelet incubating lid of claim 10, further comprising a panel having a display configured to show temperature of the heated lid portion and buttons configured to allow an operator to select a desired temperature for the heated lid portion.

13. The platelet incubating lid of claim 12, further comprising a controller configured to provide power and temperature control to the heated plate.

14. The platelet incubating lid of claim 13, wherein the panel comprises a user interface mechanism for the controller.

15. The platelet incubating lid of claim 13, further comprising a temperature logger configured to keep a record of temperature of the heated lid.

16. The platelet incubating lid of claim 15, wherein the record of temperature of the heated lid is retrievable through a portable digital storage device or a wireless network connection.

17. The platelet incubating lid of claim 15, wherein the temperature logger is integrated into the controller.

18. An apparatus, comprising:
a portable refrigeration unit; and
a platelet incubating lid mounted onto the portable refrigeration unit,
wherein the platelet incubating lid comprises:
a base and;
a platelet incubator having a heating plate and a transparent cover.

19. The apparatus of claim 18, further comprising a temperature logger configured to keep a record of temperature of the platelet incubator.

20. The platelet incubating lid of claim 19, wherein the record of temperature of the platelet incubator is retrievable through a portable digital storage device or a wireless network connection.
